(19) United States
(12) Patent Application Publication
(10) Pub. No.: US 2025/0295721 A1
(43) Pub. Date: Sep. 25, 2025

(54) STABLE RELEASE NUTRITIONAL TARGETING COMPOSITION AND ITS USE

(71) Applicant: Shenzhen Fushan Biotech Co., Ltd., Shenzhen (CN)

(72) Inventors: Xiaolong LI, Shenzhen (CN); Zhipeng ZHU, Shenzhen (CN)

(21) Appl. No.: 18/609,554

(22) Filed: Mar. 19, 2024

(51) Int. Cl. A61K 36/31; A61K 9/20; A61K 31/702

(52) U.S. Cl. CPC A61K 36/31; A61K 9/2009; A61K 31/702

(57) ABSTRACT

The invention relates to a stable release nutritional targeting composition comprising glucoraphanin, myrosinase and a calcium salt, which can smoothly release sulforaphane. The invention also relates to a method for converting glucoraphanin into sulforaphane in vitro comprising steps of providing the composition and mixing the composition with water or an aqueous solution.

### FIELD

The invention belongs to the field of biomedicine, in particular to a stable release nutritional targeting composition, and the invention also relates to use of the composition in the manufacture of a product for preventing and/or treating diseases or conditions which can be prevented and/or treated by using sulforaphane.

### BACKGROUND

According to the World Health Organization's 2014 annual report, cancer is the leading cause of morbidity and mortality worldwide, posing a huge threat to people's lives and health, and bringing a heavy economic burden to social development. In the long process of fighting cancer, the concept of cancer chemoprevention was proposed in 1976, which refers to the strategy of using natural or synthetic chemicals to prevent, slow down or reverse the development of cancer.

One idea of cancer chemoprevention is nutritional targeting. One of the main ideas of nutritional targeting is to activate the Nrf2 pathway (i.e. NF-E2-related factor 2, a transcription factor that regulates antioxidant stress response) by using the active components of dietary supplements (such as broccoli) to promote the expression of phase II enzymes. The effects can be related not only to the chemoprevention of cancer, but also to the treatment and prevention of gastric ulcer, Helicobacter pylori infection and antioxidation, as well as the prevention and/or treatment of diabetes, a cardiovascular disease, Helicobacter pylori infection, autism, schizophrenia, depression, Alzheimer's disease, etc.

Sulforaphane (SFN), with chemical name 1-isothiocyanate-4-methanesulfonylbutane, belonging to an isothiocyanate, is a biologically active substance found by Paul Talalay of John Hopkins University from broccoli that has the ability to prevent cancer. It is the strongest anti-cancer ingredient found in vegetables to date. The molecular mechanism of sulforaphane and the results of cell experiments further show that sulforaphane functions as cancer chemoprevention by regulating the phase II enzyme activity for metabolic detoxification of phase I enzyme metabolites or foreign substances (Myzak M C, Dashwood R H. Cancer Lett., 2006, 233:208-18.). It is known that sulforaphane is an inducer of Nrf2 (NF-E2 related factor 2, a transcription factor regulating cell oxidative stress). The main mechanism of action is activation of Nrf2 signaling pathway and induction of phase II enzyme (NQO1, glutathione thiotransferase, γ-glutamylcysteine synthetase, glucuronyltransferase, etc.) expression, and regulation of antioxidant response elements and the like. The prior art discloses various activities and effects of sulforaphane and its precursor compound glucoraphanin, such as chemical protective agents against gastric ulcers and Helicobacter pylori infection (CN1935003A; WO2006/118941). Nrf2 is known to be a transcription factor that regulates the expression of many detoxifying enzymes and antioxidant enzymes. It is known that sulforaphane and glucoraphanin have antimicrobial activities against Gram-positive bacteria and Gram-negative bacteria and yeast. Furthermore, they have been shown to exert a protective effect on Parkinson's disease (in a mouse model) and they in particular also have diuretic, anti-anemia and laxative properties. A molecular basis investigation of the mechanism of action of sulforaphane indicates that sulforaphane and glucoraphanin act indirectly as antioxidants by stimulating phase II detoxification enzymes. In addition, sulforaphane and glucoraphanin, which are sulforaphane compounds, have been shown to have UV radiation protection, thereby avoiding sunburn, degradation caused by ROS (reactive oxygen species) and skin cancer (Talalay P; Fahey J W; Healy Z R; Wehage S L; Benedict A L; Min C.; Dinkova-Kostova A T PNAS, 2007, 104, 17500-17505). Factor Nrf2 has been shown to play an important role in growth factor regulation, signaling and tissue repair (specifically, oxidative stress-induced liver regeneration) in recent years (Beyer T.; Xu W.; Teupser D.; Keller U.; Bugnon P.; Hildt E.; Thiery J.; Yuet Wai K.; Werner S. The EMBO Journal, 2008, 27, 212-223).

In summary, based on the above mechanism, it has been found and confirmed that sulforaphane as an isothiocyanate has various activities and functions associated with its phase II enzyme regulation and Nrf2 activation. With the deepening of research, it has been found that in addition to the role of sulforaphane in the field of cancer chemoprevention, it also has preventive and/or therapeutic effects in many other diseases including diabetes, cardiovascular disease, Helicobacter pylori infection, autism, schizophrenia, depression, and Alzheimer diseases, and it has been validated in animal and clinical trials. For example, sulforaphane can reduce hepatic glucose production in patients with type 2 diabetes and improve glycemic control (Axelsson A S, Tubbs E, Mecham B, et al. Sci Transl Med., 2017, 9 (394)); can reduce vascular inflammation and prevent TNF-α-induced adhesion of monocytes to primary epithelial cells (Nallasamy P, Si H, Babu P V, et al. J Nutr Biochem., 2014, 25 (8): 824-33.); can inhibit colonization of H. pylori in the stomach of mice and humans, and reduce infection-induced gastric inflammation (Yanaka A, Fahey J W, Fukumoto A, et al. Cancer Prev Res (Phila)., 2009, 2 (4): 353-60.); can reverse autism-related abnormal symptoms in clinical trials, including oxidative stress, low antioxidant capacity, inhibited glutathione synthesis, decreased mitochondrial function and oxidative phosphorylation, enhanced lipid peroxidation and neuroinflammation (Singh K, Connors S L, Macklin E A, et al. Proc Natl Acad Sci USA, 2014, 111 (43): 15550-5.); can improve cognitive function in patients with schizophrenia (Shiina A, Kanahara N, Sasaki T, et al. (lin Psychopharmacol Neurosci., 2015, 13 (1): 62-7.). Broccoli sprouts rich in sulforaphane have a preventive effect on depression (Zhang J C, Yao W, Dong C, et al. J Nutr Biochem., 2017, 39:134-144.). Administering sulforaphane can improve cognitive function of the acute AD mouse model induced by amyloid β-protein (Aβ) in the Y-maze and passive avoidance behavior tests (Kim H V, Kim H Y, Ehrlich H Y, et al. Amyloid., 2013, 20 (1): 7-12.).

It is known that cruciferous plants are the main source of sulforaphane and its precursor compound glucoraphanin. Broccoli is a preferred crucifer plant that provides sulforaphane and its precursor compound, glucoraphanin. Broccoli is a Brassicaceae Brassica plant. It is known that the glucoraphanin content is relatively higher in broccoli seeds and seedlings (buds). Even so, it is unrealistic to consume an effective amount of sulforaphane by eating broccoli. Therefore, it is necessary to extract the broccoli and achieve its effective biological efficacy through its extract.

However, we found in our studies that for existing nutritional targeting compositions for cancer prevention, especially for a mixture of myrosinase and glucoraphanin in solid form (including raw materials in the form of broccoli and/or their extracts), the release of sulforaphane from aqueous solution is unstable and the release curve has obvious sudden release effect, resulting in isothiocyanate produced has great irritation to the stomach and intestines, which seriously affects the acceptability of the product.

Therefore, in the prior art, it is necessary to overcome the unstable release of existing nutritional targeting compositions, especially the unstable release of compositions containing glucoraphanin and myrosinase in an aqueous solution. There is an urgent need to provide a composition containing glucoraphanin and myrosinase that can stably release sulforaphane in aqueous solution.

### SUMMARY

The present inventors have unexpectedly found in the studies that if a calcium salt is added to a composition comprising glucoraphanin and myrosinase, the release stability of the composition can be well improved, and a relatively stable release curve can be obtained.

Based on this finding, in a first aspect of the invention, a composition is provided, which comprises the following components:
1) component 1 providing glucoraphanin;
2) component 2 providing myrosinase; and
3) a calcium salt.

In the present invention, the component 1 providing glucoraphanin may be any substance or raw material capable of providing a source of glucoraphanin compound. Preferably, the component 1 providing glucoraphanin is selected from the group consisting of cruciferous plants, extracts thereof and mixtures thereof. The cruciferous plant is preferably selected from the group consisting of broccoli, cauliflower, red cabbage, brussels sprouts or cabbage, wherein broccoli is particularly preferred. The crucifer plant may be whole or a part of a plant, such as a whole plant, an aerial part thereof, a flower ball, a seedling, a seed, or a combination thereof. The component 1 providing glucoraphanin may also be an extract of a cruciferous plant, such as a solvent extract, preferably an aqueous extract, an alcohol extract, a water-alcohol extract. In addition to plant tissues, extracts and mixtures thereof of cruciferous plants, the component 1 providing glucoraphanin of the present invention may further comprise chemically synthesized, semi-chemically synthesized, and/or enzymatically synthesized glucoraphanin.

In the present invention, the component 2 providing myrosinase may be any substance or raw material capable of providing a source of myrosinase. Preferably, the component 2 providing myrosinase is selected from the group consisting of cruciferous plants, extracts thereof, and mixtures thereof. Preferably, the component 2 providing myrosinase is selected from the group consisting of horseradish, radish and kale. In some preferred embodiments, the component 2 providing myrosinase is selected from the group consisting of horseradish extract, radish extract, and cabbage extract; in other preferred embodiments, the component 2 providing myrosinase is a juice or slurry from horseradish, radish, and/or kale, or a powder obtained by drying the juice or slurry.

In the present invention, broccoli means all or a part of a broccoli plant. Preferably, the broccoli is selected from the edible parts of the usual meaning; more preferably, the broccoli is selected from the group consisting of broccoli flower bulb, broccoli seed and broccoli seedling, and combinations thereof.

In the present invention, the broccoli extract means an extract of all or a part of the broccoli plant, including but not limited to an extract of broccoli, the broccoli flower bulb, the broccoli seed and/or an of broccoli seedling. On the other hand, the extract is an extract obtained by extraction with a solvent, and the extract is preferably an aqueous extract, an alcohol extract or a water-alcohol extract, and particularly preferably an aqueous extract.

In the present invention, the component 1 providing glucoraphanin is preferably selected from the group consisting of broccoli flower bulbs, broccoli seeds, broccoli seedlings, broccoli extracts, and mixtures thereof.

In the present invention, the term “calcium salt” or “calcium salt compound” refers to a salt containing calcium ion. In the invention, the calcium salt may be a calcium salt of an organic acid or a calcium salt of an inorganic acid. Exemplary calcium salts include calcium carbonate, calcium phosphate, calcium sulfate, calcium citrate, calcium tartrate, calcium sulfonate, calcium malate, calcium lactate and their mixtures. In the invention, a preferred calcium salt is selected from the group consisting of calcium carbonate, calcium citrate and calcium sulfate; more preferably, the calcium salt is selected from the group consisting of calcium carbonate and calcium sulfate; and calcium carbonate is the most preferred.

In the present invention, the mass ratio of the component 1, the component 2 and the calcium salt is (10-90):(1-90):(0.1-2); preferably (10-60):(1-60):(0.1-1), more preferably (10-40):(1-20):(0.1-0.5).

In some embodiments, the composition of the present invention consists of the following components:
1) component 1 providing glucoraphanin;
2) component 2 providing myrosinase; and
3) a calcium salt.

In some embodiments, the composition of the present invention may further comprise ascorbic acid.

The composition of the present invention is preferably in a solid form, for example, in the form of a preparation of powder, granule, capsule or tablet. More preferably, in the composition of the present invention, the component 1, the component 2 and the calcium salt are all present in solid form. For example, the component 1 may be in the form of powder (including lyophilized powder) of the extract, seedling or seed.

In some preferred embodiments, the component 1 of the invention is selected from the group consisting of broccoli seed extract, broccoli seedling powder, broccoli flower bulb lyophilized powder, and mixtures thereof.

In another aspect, the invention provides use of the composition of the invention in the manufacture of a product for prevention and/or treatment of a disease or disorder that can be prevented and/or treated by sulforaphane. The product can be a medicament or food. Preferably, the product is a medicament. In some preferred embodiments, the disease or disorder that can be prevented and/or treated by sulforaphane is selected from the group consisting of cancer, diabetes, a cardiovascular disease, Helicobacter pylori infection, autism, schizophrenia, depression, Alzheimer disease (AD) and pulmonary fibrosis.

In another aspect of the invention, a method of converting glucoraphanin to sulforaphane in vitro is provided, which comprises the following steps:
1) providing a composition according to the present invention, and
2) mixing the composition with water or an aqueous solution.

In a third aspect of the invention, a method of supplementing sulforaphane to a subject in need thereof is provided, comprising administering to the subject a composition of the present invention.

On the other hand, the invention also provides a method for improving release stability of sulforaphane in an aqueous solution, comprising step of mixing sulforaphane with a calcium salt, or comprising step of mixing a sample containing component 1 providing glucoraphanin and component 2 providing myrosinase with a calcium salt.

In another aspect, the invention also provides the use of a calcium salt in the preparation of a product for improving release stability of sulforaphane in an aqueous solution.

The present inventors unexpectedly found via studies that if a calcium salt is added to a composition comprising glucoraphanin and myrosinase, the release stability of the composition can be well improved, and a relatively stable release curve can be obtained, and the sudden release phenomenon of sulforaphane can be effectively overcome.

The present application comprises the following technical embodiments.

1. A composition, comprising the following components:
1) component 1 providing glucoraphanin,
2) component 2 providing myrosinase, and
3) a calcium salt.

2. The composition of Embodiment 1, wherein the component 1 is selected from the group consisting of a cruciferous plant, an extract thereof and a mixture thereof.

3. The composition of Embodiment 1 or 2, wherein the cruciferous plant is broccoli.

4. The composition of Embodiment 2 or 3, wherein the cruciferous plant is selected from the group consisting of whole of the plant, a part of the plant and a mixture thereof.

5. The composition of any one of Embodiments 2 to 4, wherein the cruciferous plant is selected from the group consisting of flower bulbs, seeds, sprouts and a mixture thereof.

6. The composition according to any one of Embodiments 1 to 5, wherein the mass ratio of component 1, component 2 and the calcium salt is (10-90):(1-90):(0.1-2).

7. The composition of any one of Embodiments 1 to 6, wherein the calcium salt is selected from the group consisting of organic calcium salts and inorganic calcium salts, preferably calcium carbonate, calcium citrate and calcium sulphate, more preferably calcium carbonate.

8. The composition of any one of Embodiments 1 to 7, further comprising a functional oligosaccharide.

9. The composition according to any one of Embodiments 1 to 8, wherein the functional oligosaccharide is selected from the group consisting of fructo-oligosaccharide, soy oligosaccharide, galacto-oligosaccharide, xylo-oligosaccharide, isomaltooligosaccharide, lactulooligosaccharide, cyclodextrin, chito-oligosaccharide, inulin and combinations thereof; especially selected from the group consisting of fructo-oligosaccharide, xylo-oligosaccharide, galacto-oligosaccharide, lactulooligosaccharide and isomaltooligosaccharide; particularly preferred xylo-oligosaccharide and fructo-oligosaccharide, with xylo-oligosaccharide being most preferred.

10. The composition of any one of Embodiments 1 to 9, which is in solid form.

11. The composition according to any one of Embodiments 1 to 10, which is in the form of powder, granule, capsule or tablet.

12. The composition according to any one of Embodiments 1 to 11, wherein the component 1 is selected from the group consisting of broccoli seed extract, broccoli sprout powder, broccoli curd lyophilized powder and a mixture thereof, and glucoraphanin from chemical synthesis or biosynthesis.

13. The composition according to any one of Embodiments 1 to 12, wherein the component 2 is selected from the group consisting of horseradish, radish, kale, a juice or slurry thereof, and an extract thereof.

14. A method of preventing or treating a disease or condition that can be prevented or treated with sulforaphane, comprising administering a composition of Embodiment 1 to a subject in need thereof.

15. The method of claim 14, wherein the disease or condition that can be prevented or treated with sulforaphane is selected from the group consisting of cancer, diabetes, a cardiovascular disease, Helicobacter pylori infection, autism, schizophrenia, depression, Alzheimer's disease and pulmonary fibrosis.

16. A method for converting glucoraphanin into sulforaphane in vitro, comprising steps of:
1) providing a composition according to any one of Embodiments 1 to 15,
2) mixing the composition with water or an aqueous solution.

17. A method of supplementing sulforaphane to a subject in need thereof, comprising administering to the subject a composition according to any one of Embodiments 1 to 15.

18. A method for improving smoothness of release of sulforaphane in an aqueous solution, comprising step of mixing sulforaphane with a calcium salt, or comprising step of mixing a sample containing component 1 providing glucoraphanin and component 2 providing myrosinase with a calcium salt.

19. Use of a calcium salt in the preparation of a product for improving smoothness of release of sulforaphane in an aqueous solution.

### BRIEF DESCRIPTION OF DRAWINGS

FIG. 1 is a bar chart showing the release of sulforaphane at various time periods from aqueous solution of the composition (powder) of the present invention and the control compositions 1-3; and

FIG. 2 is a bar chart showing the release of sulforaphane at various time periods from aqueous solution of the composition (tablet) of the present invention and the control compositions 1-3.

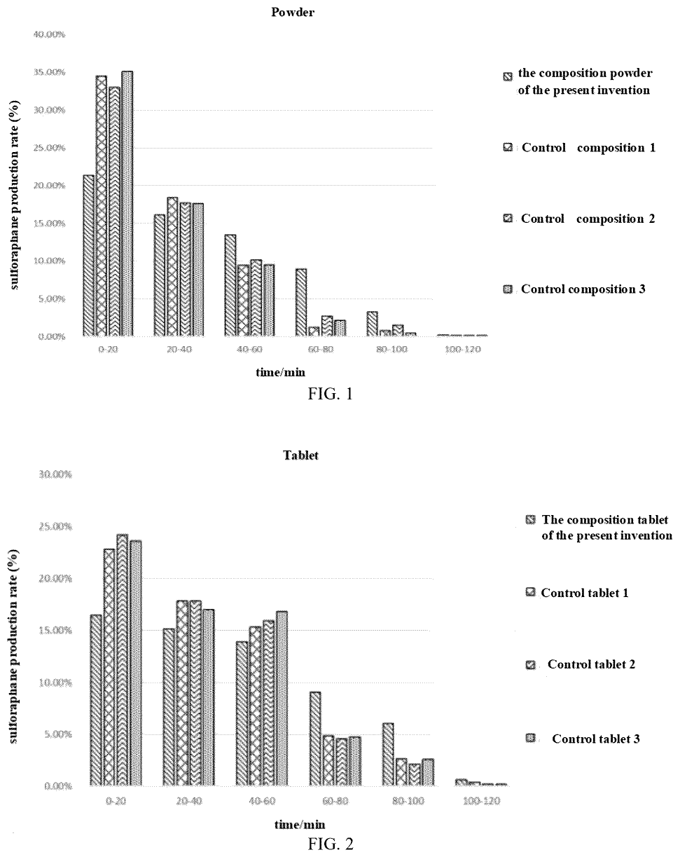

### DETAILED DESCRIPTION OF THE INVENTION

### Preparation Example 1: Preparation of the Composition of the Present Invention (Powder) and Control Compositions 1-3

Preparation of the composition of the present invention: 400 g of aqueous extract of broccoli seeds (containing 13.0% of glucoraphanin, from Brassica Protection Products LLC, USA, hereinafter the same) was mixed with 100 g of horseradish powder and 10 g of calcium carbonate, to obtain 0.51 kg of the composition, of which 10.19% was glucoraphanin. The above composition was dispensed into sachet packages at a weight of 5 g per sachet to obtain the corresponding powder product (the composition of the present invention).

Preparation of control composition 1:400 g of broccoli seed aqueous extract (containing 13.0% of glucoraphanin) was mixed with 100 g of horseradish powder and 10 g of sodium carbonate to obtain 0.51 kg of control composition 1, of which glucoraphanin accounted for 10.19%. The above control composition 1 was dispensed into sachet packages at a weight of 5 g per sachet to obtain the corresponding powder product (control composition 1).

Preparation of control composition 2:400 g of broccoli seed aqueous extract (containing 13.0% of glucoraphanin) was mixed with 100 g of horseradish powder and 10 g of potassium carbonate to obtain 0.51 kg of control composition 2, in which glucoraphanin accounted for 10.19%. The above control composition 2 was dispensed into sachet packages at a weight of 5 g per sachet to obtain the corresponding powder product (control composition 2).

Preparation of control composition 3:400 g of broccoli seed aqueous extract (containing 13.0% of glucoraphanin) was mixed with 100 g of horseradish powder and 10 g of ammonium carbonate to obtain 0.51 kg of control composition 3, in which glucoraphanin accounted for 10.19%. The above control composition 3 was dispensed into sachet packages at a weight of 5 g per sachet to obtain the corresponding powder product (control composition 3).

### Example 2: Determination of In Vitro Release Profiles of Powder

1 g of each of the above four powder products was dissolved in 30 ml of artificial gastric fluid (prepared with pH adjusted to 3.5 according to the Chinese Pharmacopoeia, 2015 edition, for simulating the condition of postprandial gastric fluid) at 37° C. for the reaction, and the samples were taken at the 0^{th} min, 20^{th} min, 40^{th} min, 60^{th} min, 80^{th} min, 100^{th} min, and 120^{th} min, respectively, and the content of sulforaphane in aqueous solution was determined by HPLC and the production rate of sulforaphane was calculated.

HPLC method for the determination of sulforaphane: the sample solution was taken and passed through a 0.45 μm filter membrane, and then analyzed by HPLC. HPLC conditions: chromatographic column: Unitary C18 (4.6 mm×250 mm, 5 μm) of Huapu Company; column temperature: 30° C.; mobile phase: 70% water-30% acetonitrile; flow rate: 0.8 mL/min; injection volume: 10 μL; UV detection wavelength: 245 nm.

The experimental results are shown in Table 1 below.

TABLE 1
Comparison of sulforaphane release of the composition of the
present invention and control composition powder products 1-3

| | sulforaphane production rate (%) | | | | | | |
|---|---|---|---|---|---|---|---|
| group | 0 min | 20 min | 40 min | 60 min | 80 min | 100 min | 120 min |
| the composition | 0% | 21.3% | 37.4% | 50.8% | 59.7% | 62.9% | 63.2% |
| of the present | | | | | | | |
| invention | | | | | | | |
| control | 0% | 34.4% | 52.8% | 62.2% | 63.4% | 64.1% | 64.3% |
| composition 1 | | | | | | | |
| control | 0% | 32.9% | 50.6% | 60.7% | 63.4% | 64.9% | 65.0% |
| composition 2 | | | | | | | |
| control | 0% | 35.0% | 52.6% | 62.0% | 64.1% | 64.6% | 64.7% |
| composition 3 | | | | | | | |

From the experimental results, it can be seen that the composition of the present invention containing calcium salt had smooth release characteristics in aqueous solution and did not exhibit the sudden release phenomenon presented by the control compositions, as compared to the control compositions 1-3 without a calcium salt, which suggests that the release of sulforaphane from the mixtures is more smooth with the addition of a calcium salt. In particular, the composition of the present invention clearly overcame the problem of too rapid release of the control compositions in the first 20 minutes, thus significantly reducing the gastric irritation of the product when taken, which was an unexpected result.

### Preparation Example 2: Preparation of the Composition (Tablet) of the Present Invention and its Control Tablets 1-3

(1) Preparation of the composition (tablet) of the present invention: 200 g of aqueous extract of broccoli sprouts (containing 13.0% of glucoraphanin) was mixed with 50 g of horseradish powder, 5 g of calcium carbonate, and 250 g of other tablet-pressing excipients (starch, maltodextrin, and hydroxypropylmethylcellulose, with the ratio (w/w) of 5:80:2, hereinafter the same), and then pressed into tablets with a tablet weight of 1 g/tablet, and coated with film coating, to obtain approximately 0.505 kg of the tablets of the present invention, in which glucoraphanin accounted for 5.15%. The above tablets of the present invention were packed into bottles of 60 tablets per bottle, and the bottles were sealed with a desiccant to obtain the corresponding tablet products.

(2) Preparation of control tablet 1:200 g of aqueous extract of broccoli sprouts (containing 13.0% of glucoraphanin) was mixed with 50 g of horseradish powder, 5 g of sodium carbonate and 250 g of other tablet-pressing excipients, and then pressed according to the tablet weight of 1 g/tablet, coated with film coating, to obtain about 0.505 kg of the control tablet 1, in which glucoraphanin accounted for 5.15%. The above control tablet 1 was divided into bottles of 60 tablets per bottle and sealed with a desiccant to obtain the corresponding tablet product.

(3) Preparation of control tablet 2:200 g of aqueous extract of broccoli sprouts (containing 13.0% of glucoraphanin) was mixed with 50 g of horseradish powder, 5 g of potassium carbonate and 250 g of other tablet-pressing excipients, and then pressed according to the tablet weight of 1 g/tablet and coated with film coating, to obtain about 0.505 kg of the control tablet 2, in which glucoraphanin accounted for 5.15%. The above control tablet 2 is divided into bottles of 60 tablets per bottle and sealed with a desiccant to obtain the corresponding tablet product.

(4) Preparation of control tablet 3:200 g of aqueous extract of broccoli sprouts (containing 13.0% of glucoraphanin) was mixed with 50 g of horseradish powder, 5 g of ammonium carbonate and 250 g of other tablet-pressing excipients, and then pressed according to a tablet weight of 1 g/tablet, coated with film coating, and about 0.505 kg of control tablet 3 was obtained, in which glucoraphanin accounted for 5.15%. The above control tablet 3 was divided into bottles of 60 tablets per bottle and sealed with a desiccant to obtain the corresponding tablet product.

### Example 3: Determination of In Vitro Release Profiles of Tablets

2 g of each of the above four tablet products prepared in Preparation Example 2 was dissolved in 30 ml of artificial gastric fluid (prepared with pH adjusted to 3.5 according to the Chinese Pharmacopoeia, 2015 edition, for simulating the condition of postprandial gastric fluid) at 37° C. for the reaction, and the samples were taken at the 0^{th} min, 20^{th} min, 40^{th} min, 60^{th} min, 80^{th} min, 100^{th} min, and 120^{th} min, respectively, and the content of sulforaphane in aqueous solution was determined by HPLC and the production rate of sulforaphane was calculated.

HPLC method for the determination of sulforaphane: the sample solution was taken and passed through a 0.45 μm filter membrane, and then analyzed by HPLC. HPLC conditions: chromatographic column: Unitary C18 (4.6 mm×250 mm, 5 μm) of Huapu Company; column temperature: 30° C.; mobile phase: 70% water-30% acetonitrile; flow rate: 0.8 mL/min; injection volume: 10 μL; UV detection wavelength: 245 nm.

The experimental results are shown in Table 2 below.

TABLE 2
Comparison of sulforaphane release of the composition tablet
of the present invention and control tablets 1-3

| | sulforaphane production rate (%) | | | | | | |
|---|---|---|---|---|---|---|---|
| group | 0 min | 20 min | 40 min | 60 min | 80 min | 100 min | 120 min |
| the composition | 0.00% | 16.43% | 31.58% | 45.45% | 54.53% | 60.53% | 61.13% |
| tablet of the present | | | | | | | |
| invention | | | | | | | |
| control tablet 1 | 0.00% | 22.80% | 40.58% | 55.88% | 60.68% | 63.30% | 63.68% |
| control tablet 2 | 0.00% | 24.15% | 41.93% | 57.83% | 62.33% | 64.43% | 64.58% |
| control tablet 3 | 0.00% | 23.55% | 40.50% | 57.30% | 62.03% | 64.58% | 64.65% |

From the experimental results, it can be seen that the tablet compositions of the present invention containing a calcium salt had smooth release characteristics in aqueous solution compared to the control tablets 1-3 without a calcium salt, and did not exhibit the sudden release phenomenon presented by the control compositions, which suggested that sulforaphane was released more smoothly from the product with the addition of a calcium salt. In particular, the tablet compositions of the present invention clearly overcome the problem of too rapid release of the control tablet compositions in the first 20 minutes, and thus can considerably reduce the gastric irritation of the product when taken, and such a result is unexpected.

It should be understood that while the present invention is specifically disclosed by way of preferred embodiments, those skilled in the art can adopt optional features, modifications, improvements and variations of the embodiments of the present invention described in the disclosures herein, considering such modifications, improvements and variations to be within the scope of the present invention. The materials, methods and embodiments provided herein as exemplary or preferred embodiments are exemplary and are not intended to limit the scope of the present invention.

1. A composition, comprising the following components:
1) component 1 providing glucoraphanin,
2) component 2 providing myrosinase, and
3) a calcium salt.

2. The composition of claim 1, wherein the component 1 is selected from the group consisting of a cruciferous plant, an extract thereof and a mixture thereof.

3. The composition of claim 1, wherein the cruciferous plant is broccoli.

4. The composition of claim 2, wherein the cruciferous plant is selected from the group consisting of whole of the plant, a part of the plant and a mixture thereof.

5. The composition of claim 2, wherein the cruciferous plant is selected from the group consisting of flower bulbs, seeds, sprouts and a mixture thereof.

6. The composition of claim 1, wherein the mass ratio of component 1, component 2 and the calcium salt is (10-90):(1-90):(0.1-2).

7. The composition of claim 1, wherein the calcium salt is selected from the group consisting of organic calcium salts and inorganic calcium salts, preferably calcium carbonate, calcium citrate and calcium sulphate, more preferably calcium carbonate.

8. The composition of claim 1, further comprising a functional oligosaccharide.

9. The composition of claim 1, wherein the functional oligosaccharide is selected from the group consisting of fructo-oligosaccharide, soy oligosaccharide, galacto-oligosaccharide, xylo-oligosaccharide, isomaltooligosaccharide, lactulooligosaccharide, cyclodextrin, chito-oligosaccharide, inulin and combinations thereof; especially selected from the group consisting of fructo-oligosaccharide, xylo-oligosaccharide, galacto-oligosaccharide, lactulooligosaccharide and isomaltooligosaccharide; particularly preferred xylo-oligosaccharide and fructo-oligosaccharide, with xylo-oligosaccharide being most preferred.

10. The composition of claim 1, which is in solid form.

11. The composition of claim 1, which is in the form of powder, granule, capsule or tablet.

12. The composition of claim 1, wherein the component 1 is selected from the group consisting of broccoli seed extract, broccoli sprout powder, broccoli curd lyophilized powder and a mixture thereof, and glucoraphanin from chemical synthesis or biosynthesis.

13. The composition of claim 1, wherein the component 2 is selected from the group consisting of horseradish, radish, kale, a juice or slurry thereof, and an extract thereof.

14. A method of preventing or treating a disease or condition that can be prevented or treated with sulforaphane, comprising administering a composition of claim 1 to a subject in need thereof.

15. The method of claim 14, wherein the disease or condition that can be prevented or treated with sulforaphane is selected from the group consisting of cancer, diabetes, a cardiovascular disease, Helicobacter pylori infection, autism, schizophrenia, depression, Alzheimer's disease and pulmonary fibrosis.

16. A method for converting glucoraphanin into sulforaphane in vitro, comprising steps of:
1) providing a composition according to claim 1, and
2) mixing the composition with water or an aqueous solution.

17. A method of supplementing sulforaphane to a subject in need thereof, comprising administering to the subject a composition according to claim 1.
